(19) United States
(12) Patent Application Publication
(10) Pub. No.: US 2010/0267713 A1
(43) Pub. Date: Oct. 21, 2010

(54) 4,4-DISUBSTITUTED PIPERIDINES

(76) Inventors: Peter Herold, Basel (CH); Stjepan Jelakovic, Basel (CH); Robert Mah, Basel (CH); Christiane Marti, Basel (CH); Stefan Stutz, Basel (CH); Vincenzo Tschinke, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/740,720

(22) PCT Filed: Oct. 31, 2008
(86) PCT No.: PCT/EP2008/064767

§ 371 Date: Apr. 30, 2010

(30) Foreign Application Priority Data
Nov. 2, 2007 (EP) 0719884.0

(51) Int. Cl. A61K 31/538; C07D 265/36; A61P 9/12; A61P 9/10; A61P 27/06

(52) U.S. Cl. 514/230.5; 544/105

(57) ABSTRACT

The application relates to 4,4-disubstituted piperidines of the general formula (I) and their salts, preferably their pharmaceutically acceptable salts, in which R², has the meanings explained in the description, a process for their preparation and the use of these compounds as medicines, especially as renin inhibitors.

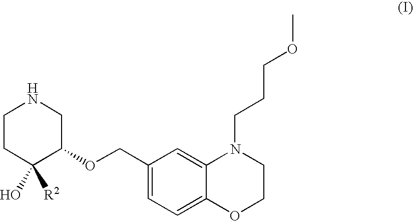

### FIELD OF THE INVENTION

The present invention relates to novel 4,4-disubstituted piperidines, process for their preparation and the use of the compounds as medicines, in particular as renin inhibitors.

### BACKGROUND OF THE INVENTION

Piperidine derivatives for use as medicines are disclosed for example in WO 97/09311. Furthermore, piperidine derivatives showing a 3,4-dihydro-2H-benzo[1,4]oxazin-6-ylmethoxy-substituent are disclosed in WO 2007/082907 and WO 2006/103277. However, especially with regard to renin inhibition, there continues to be a need for highly potent active ingredients. The main focus is in improving the pharmacokinetic properties. These properties, which are directed at better bioavailability, are for example absorption, metabolic stability, solubility or lipophilicity.

### DETAILED DESCRIPTION OF THE INVENTION

The invention therefore relates firstly to substituted piperidines of the general formula

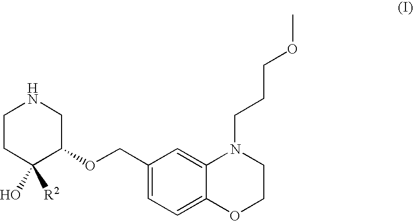

and their salts, preferably their pharmaceutically acceptable salts, in which
R² is pyridyl, which is substituted by 1-3 radicals selected independently from the group consisting of
C₁₋₆-alkanoyloxy-C₁₋₆-alkyl,
C₂₋₆-alkenyl,
C₂₋₆-alkenyloxy,
C₂₋₆-alkenyloxy-C₁₋₆-alkyl,
C₁₋₆-alkoxy,
C₁₋₆-alkoxy-C₁₋₆-alkoxy,
C₁₋₆-alkoxy-C₁₋₆-alkoxy-C₁₋₆-alkoxy,
C₁₋₆-alkoxy-C₁₋₆-alkoxy-C₁₋₆-alkoxy-C₁₋₆-alkyl,
C₁₋₆-alkoxy-C₁₋₆-alkoxy-C₁₋₆-alkyl,
C₁₋₆-alkoxy-C₁₋₆-alkyl,
C₁₋₆-alkoxy-C₁₋₆-alkylamino-C₁₋₆-alkyl,
C₁₋₆-alkoxy-C₁₋₆-alkylsulfanyl,
C₁₋₆-alkoxy-C₁₋₆-alkylsulfanyl-C₁₋₆-alkyl,
C₁₋₆-alkoxycarbonyl,
C₁₋₆-alkoxycarbonyloxy-C₁₋₆-alkyl,
C₁₋₆-alkyl,
C₁₋₆-alkylsulfanyl,
C₁₋₆-alkylsulfanyl-C₁₋₆-alkoxy,
C₁₋₆-alkylsulfanyl-C₁₋₆-alkoxy-C₁₋₆-alkyl,
C₁₋₆-alkylsulfanyl-C₁₋₆-alkyl,
C₁₋₆-alkylsulfonyl-C₁₋₆-alkoxy-C₁₋₆-alkyl,
C₁₋₆-alkylsulfonyl-C₁₋₆-alkyl,
C₂₋₈-alkynyl,
optionally N-mono- or N,N-di-C₁₋₆-alkylated amino-C₁₋₆-alkoxy,
optionally N-mono- or N,N-di-C₁₋₆-alkylated amino-carbonyl-C₁₋₆-alkyl,
aryl-pyrrolidinyl-C₀₋₆-alkoxy,
heterocyclyl-pyrrolidinyl-C₀₋₆-alkoxy,
aryloxy,
aryl-C₀₋₆-alkoxy-C₁₋₆-alkoxy,
aryl-C₀₋₆-alkoxy-C₁₋₆-alkoxy-C₁₋₆-alkyl,
carboxy-C₁₋₆-alkyl,
cyano,
cyano-C₁₋₆-alkyl,
C₃₋₈-cycloalkyl-C₀₋₆-alkoxy-C₁₋₆-alkoxy,
C₃₋₈-cycloalkyl-C₀₋₆-alkoxy-C₁₋₆-alkoxy-C₁₋₆-alkyl,
C₃₋₈-cycloalkyl-C₀₋₆-alkoxy-C₁₋₆-alkyl,
C₃₋₈-cycloalkyl-C₀₋₆-alkylamino-C₁₋₆-alkyl,
heterocyclyl-carbonyl-C₁₋₆-alkyl,
heterocyclyl-C₁₋₆-alkyl,
heterocyclyl-sulfanyl-C₁₋₆-alkoxy-C₁₋₆-alkyl and
heterocyclyl-C₀₋₆-alkoxy-C₁₋₆-alkyl;
and may, in addition to the aforementioned substituents, also be substituted by a maximum of 2 halogens, the maximum total number of substituents on the pyridyl radical of R² being 3.

The meaning of “C₀-alkyl” in the above (and hereinafter) mentioned C₀₋₆-alkyl groups is a bond or, if located at a terminal position, a hydrogen atom.

The meaning of “C₀-alkoxy” in the above (and hereinafter) mentioned C₀₋₆-alkoxy groups is “—O—” or, if located at a terminal position, an —OH group.

C₁₋₆-Alkyl and alkoxy radicals may be linear or branched. Examples of C₁₋₆-alkyl and alkoxy radicals are methyl, ethyl, n-propyl, isopropyl, n-butyl, isobutyl, sec-butyl, tert-butyl, pentyl, hexyl, and methoxy, ethoxy, propoxy, isopropoxy, butoxy, isobutoxy, sec-butoxy and tert-butoxy. C₁₋₆-Alkylenedioxy radicals are preferably methylenedioxy, ethylenedioxy and propylenedioxy. C₁₋₆-alkanoyl refers to C₁₋₆-alkylcarbonyl. Examples of C₁₋₆-alkanoyl radicals are acetyl, propionyl and butyryl.

Cycloalkyl refers to a saturated cyclic hydrocarbon radicals having 3 to 7 carbon atoms, for example cyclopropyl, cyclobutyl or cyclopentyl.

C₁₋₆-Alkylene radicals may be linear or branched and are, for example, methylene, ethylene, propylene, 2-methylpropylene, 2-methylbutylene, 2-methylpropyl-2-ene, butyl-2-ene, butyl-3-ene, propyl-2-ene, tetra-, penta- and hexamethylene; C₂₋₆-alkenylene radicals are, for example, vinylene and propenylene; C₂₋₆-alkynylene radicals are, for example, ethynylene; acyl radicals are alkanoyl radicals, preferably C₁₋₆-alkanoyl radicals, or aroyl radicals such as benzoyl.

Aryl refers to mononuclear aromatic radicals which may be substituted one or more times, such as, for example, phenyl or substituted phenyl, and may be unsubstituted or substituted one or more times, e.g. substituted once or twice by C₁₋₆-alkoxy, C₁₋₆-alkyl, optionally esterified carboxy, cyano, halogen, hydroxy, halogen substituted C₁₋₆-alkoxy, halogen substituted C₁₋₆-alkyl or phenyl.

The term substituted by halogen refers to a substituent such as bromo, chloro, fluoro or iodo.

The term heterocyclyl refers to 3-7 membered monocyclic, saturated, partially unsaturated and maximally unsaturated heterocyclic radicals having 1 to 5 nitrogen and/or 1 or 2 sulfur or oxygen atoms, which may be substituted one or more times, such as, for example, substituted once, twice or three times by C₁₋₆-alkoxy, C₁₋₆-alkoxy-C₁₋₆-alkyl, C₁₋₆-alkyl, aryl, cyano, halogen, heterocyclyl, hydroxy, halogen substituted C₁₋₆-alkoxy or halogen substituted C₁₋₆-alkyl. Heterocyclyl radicals which comprise a nitrogen atom may be linked either via the N atom or via a C atom to the remainder of the molecule.

Examples of such heterocycles are

imidazolyl,
oxetanyl,
pyrazolyl,
pyrrolidinyl,
tetrazolyl,
thiazolyl,
triazolyl.

Heterocyclyl radicals which comprise a nitrogen atom may be linked either via the N atom or via a C atom to the remainder of the molecule.

Hydroxy-substituted C₁₋₆-alkoxy may be for example hydroxy-C₁₋₆-alkoxy or else polyhydroxy-C₁₋₆-alkoxy.

The term halogen-substituted C₁₋₆-alkyl refers to C₁₋₆-alkyl radicals which may be substituted by 1-6 halogen atoms, such as, for example, bromo, chloro, fluoro, iodo. An analogous statement applies to radicals, such as halogen-substituted C₁₋₆-alkoxy.

Salts are primarily the pharmaceutically acceptable or nontoxic salts of compounds of formula (I). The term “pharmaceutically acceptable salts” encompasses salts with inorganic or organic acids, such as hydrochloric acid, hydrobromic acid, nitric acid, sulfuric acid, phosphoric acid, citric acid, formic acid, maleic acid, acetic acid, succinic acid, tartaric acid, methanesulfonic acid, p-toluenesulfonic acid and the like.

Salts of compounds having salt-forming groups are in particular acid addition salts, salts with bases, or, in the presence of a plurality of salt-forming groups, in some cases also mixed salts or internal salts.

Such salts are formed, for example, from compounds of formula (I) with an acidic group, for example a carboxyl or sulfonyl group, and are, for example, the salts thereof with suitable bases such as non-toxic metal salts derived from metals of group Ia, Ib, IIa and IIb of the Periodic Table of the Elements, for example alkali metal, in particular lithium, sodium, or potassium, salts, alkaline earth metal salts, for example magnesium or calcium salts, and also zinc salts and ammonium salts, including those salts which are formed with organic amines, such as optionally hydroxy-substituted mono-, di- or trialkylamines, in particular mono-, di- or tri(lower alkyl)amines, or with quaternary ammonium bases, e.g. methyl-, ethyl-, diethyl- or triethylamine, mono-, bis- or tris(2-hydroxy(lower alkyl))amines, such as ethanol-, diethanol- or triethanolamine, tris(hydroxymethyl)methylamine or 2-hydroxy-tert-butylamine, N,N-di(lower alkyl)-N-(hydroxy(lower alkyl))amine, such as N,N-di-N-dimethyl-N-(2-hydroxyethyl)amine, or N-methyl-D-glucamine, or quaternary ammonium hydroxides such as tetrabutyl ammoniumhydroxide. The compounds of formula (I) having a basic group, for example an amino group, may form acid addition salts, for example with suitable inorganic acids, e.g. hydrohalic acid such as hydrochloric acid, hydrobromic acid, sulfuric acid with replacement of one or both protons, phosphoric acid with replacement of one or more protons, e.g. orthophosphoric acid or metaphosphoric acid, or pyrophosphoric acid with replacement of one or more protons, or with organic carboxylic, sulfonic or phosphonic acids or N-substituted sulfamic acids, e.g. acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid, isonicotinic acid, and also amino acids, for example the alpha-amino acids mentioned above, and also methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, 2- or 3-phosphoglycerate, glucose 6-phosphate, N-cyclo-hexylsulfamic acid (with formation of the cyclamates) or with other acidic organic compounds such as ascorbic acid. Compounds of formula (I) having acidic and basic groups may also form internal salts.

Salts obtained may be converted to other salts in a manner known per se, acid addition salts, for example, by treating with a suitable metal salt such as a sodium, barium or silver salt, of another acid in a suitable solvent in which an inorganic salt which forms is insoluble and thus separates out of the reaction equilibrium, and base salts by release of the free acid and salt reformation.

The compounds of formula (I), including their salts, may also be obtained in the form of hydrates or include the solvent used for the crystallization.

For the isolation and purification, pharmaceutically unsuitable salts may also find use.

The compounds of formula (I) also include those compounds in which one or more atoms are replaced by their stable, non-radioactive isotopes; for example a hydrogen atom by deuterium.

The compounds of formula (I) have at least two asymmetric carbon atoms and may therefore be in the form of optically pure diastereomers, diastereomeric mixtures, diastereomeric racemates, mixtures of diastereomeric racemates or as meso compounds. The invention encompasses all of these forms. Diastereomeric mixtures, diastereomeric racemates or mixtures of diastereomeric racemates may be separated by customary procedures, for example by column chromatography, thin-layer chromatography, HPLC and the like.

The compounds of formula (I) may also be prepared in optically pure form. The separation into antipodes can be effected by procedures known per se, either preferably at an earlier synthetic stage by salt formation with an optically active acid, for example (+)- or (−)-mandelic acid and separation of the diastereomeric salts by fractional crystallization, or preferably at a relatively late stage by derivatizing with a chiral auxiliary building block, for example (+)- or (−)-camphanoyl chloride, and separation of the diastereomeric products by chromatography and/or crystallization and subsequent cleavage of the bonds to give the chiral auxiliary. The pure diastereomeric salts and derivatives may be analysed to determine the absolute configuration of the piperidine present with common spectroscopic procedures, and X-ray spectroscopy on single crystals constitutes a particularly suitable procedure.

It is possible for the configuration at individual chiral centres in a compound of formula (I) to be inverted selectively. For example, the configuration of asymmetric carbon atoms which bear nucleophilic substituents, such as amino or hydroxyl, may be inverted by second-order nucleophilic substitution, if appropriate after conversion of the bonded nucleophilic substituent to a suitable nucleofugic leaving group and reaction with a reagent which introduces the original substituents, or the configuration at carbon atoms having hydroxyl groups can be inverted by oxidation and reduction, analogously to the process in the European patent application EP-A-0 236 734. Also advantageous is the reactive functional modification of the hydroxyl group and subsequent replacement thereof by hydroxyl with inversion of configuration.

The compound groups mentioned below are not to be regarded as closed, but rather parts of these compound groups may be exchanged with one another or with the definitions given above or omitted in a sensible manner, for example to replace general by more specific definitions. The definitions are valid in accordance with general chemical principles, such as, for example, the common valences for atoms.

The compounds of formula (I) can be prepared in an analogous manner to preparation processes disclosed in the literature. Similar preparation processes are described for example in WO 97/09311 and WO 00/063173. Details of the specific preparation variants can be found in the examples.

Preference is given to compounds of the formula (I) and the salts thereof, preferably the pharmaceutically acceptable salts thereof, in which the substituted pyridyl radical R² is bonded to the remainder of the molecule at the 2-, 3-, 5- or 6-position.

Preference is also given to compounds of the formula (I) and the salts thereof, preferably the pharmaceutically acceptable salts thereof, in which R² is

pyridyl, substituted by 1-3 radicals selected independently from the group consisting of
C₁₋₆-alkoxy,
C₁₋₆-alkoxy-C₁₋₆-alkoxy,
C₁₋₆-alkoxy-C₁₋₆-alkoxy-C₁₋₆-alkoxy,
C₁₋₆-alkoxy-C₁₋₆-alkoxy-C₁₋₆-alkoxy-C₁₋₆-alkyl,
C₁₋₆-alkoxy-C₁₋₆-alkoxy-C₁₋₆-alkyl,
C₁₋₆-alkoxy-C₁₋₆-alkyl,
C₁₋₆-alkoxy-C₁₋₆-alkylsulfanyl,
C₁₋₆-alkoxy-C₁₋₆-alkylsulfanyl-C₁₋₆-alkyl,
C₁₋₆-alkyl,
C₁₋₆-alkylsulfanyl-C₁₋₆-alkoxy,
C₁₋₆-alkylsulfanyl-C₁₋₆-alkoxy-C₁₋₆-alkyl,
aryl-pyrrolidinyl-C₀₋₆-alkoxy,
C₃₋₈-cycloalkyl-C₀₋₆-alkoxy-C₁₋₆-alkyl,
heterocyclyl-C₀₋₆-alkoxy-C₁₋₆-alkyl and
heterocyclyl-pyrrolidinyl-C₀₋₆-alkoxy.

R² is particularly preferably

pyridyl, substituted by 1-2 radicals selected independently from the group consisting of
C₁₋₆-alkoxy,
C₁₋₆-alkoxy-C₁₋₆-alkoxy,
C₁₋₆-alkoxy-C₁₋₆-alkoxy-C₁₋₆-alkoxy,
C₁₋₆-alkoxy-C₁₋₆-alkoxy-C₁₋₆-alkoxy-C₁₋₆-alkyl,
C₁₋₆-alkoxy-C₁₋₆-alkoxy-C₁₋₆-alkyl,
C₁₋₆-alkyl,
C₃₋₈-cycloalkyl-C₀₋₆-alkoxy-C₁₋₆-alkyl,
heterocyclyl-C₀₋₆-alkoxy-C₁₋₆-alkyl and
heterocyclyl-pyrrolidinyl-C₀₋₆-alkoxy.

R² is very particularly preferably

pyridyl, substituted by 1 radical selected from the group consisting of
C₁₋₆-alkoxy,
C₁₋₆-alkoxy-C₁₋₆-alkoxy,
C₁₋₆-alkoxy-C₁₋₆-alkoxy-C₁₋₆-alkoxy,
C₁₋₆-alkoxy-C₁₋₆-alkoxy-C₁₋₆-alkoxy-C₁₋₆-alkyl,
C₁₋₆-alkoxy-C₁₋₆-alkoxy-C₁₋₆-alkyl,
C₁₋₆-alkyl,
C₃₋₈-cycloalkyl-C₀₋₆-alkoxy-C₁₋₆-alkyl,
heterocyclyl-C₀₋₆-alkoxy-C₁₋₆-alkyl and
heterocyclyl-pyrrolidinyl-C₀₋₆-alkoxy.

Prodrug derivatives of the compounds described herein are derivatives thereof which on in vivo use liberate the original compound by a chemical or physiological process. A prodrug may for example be converted into the original compound when a physiological pH is reached or by enzymatic conversion. Possible examples of prodrug derivatives are esters of freely available carboxylic acids, S- and O-acyl derivatives of thiols, alcohols or phenols, the acyl group being defined as herein. Preferred derivatives are pharmaceutically acceptable ester derivatives which are converted by solvolysis in physiological medium into the original carboxylic acid, such as, for example, lower alkyl esters, cycloalkyl esters, lower alkenyl esters, benzyl esters, mono- or disubstituted lower alkyl esters such as lower omega-(amino, mono- or dialkylamino, carboxy, lower alkoxycarbonyl)-alkyl esters or such as lower alpha-(alkanoyloxy, alkoxycarbonyl or dialkylaminocarbonyl)-alkyl esters; conventionally, pivaloyloxymethyl esters and similar esters are used as such.

Because of the close relationship between a free compound, a prodrug derivative and a salt compound, a particular compound in this invention also includes its prodrug derivative and salt form, where this is possible and appropriate.

The compounds of formula (I) and their pharmaceutically acceptable salts have an inhibitory effect on the natural enzyme renin. The latter passes from the kidneys into the blood and there brings about the cleavage of angiotensinogen to form the decapeptide angiotensin I which is then cleaved in the lung, the kidneys and other organs to the octapeptide angiotensin II. Angiotensin II raises the blood pressure both directly by arterial constriction, and indirectly by releasing the hormone aldosterone, which retains sodium ions, from the adrenals, which is associated with an increase in the extracellular fluid volume. This increase is attributable to the effect of angiotensin II itself or of the heptapeptide angiotensin III formed therefrom as cleavage product. Inhibitors of the enzymatic activity of renin bring about a reduction in the formation of angiotensin I and, as a consequence thereof, the formation of a smaller amount of angiotensin II. The reduced concentration of this active peptide hormone is the direct cause of the blood pressure-lowering effect of renin inhibitors.

The effect of renin inhibitors is detected inter alia experimentally by means of in vitro tests where the reduction in the formation of angiotensin I is measured in various systems (human plasma, purified human renin together with synthetic or natural renin substrate). The following in vitro test of Nussberger et al. (1987) J. Cardiovascular Pharmacol., Vol. 9, pp. 39-44, is used inter alia. This test measures the formation of angiotensin I in human plasma. The amount of angiotensin I formed is determined in a subsequent radioimmunoassay. The effect of inhibitors on the formation of angiotensin I is tested in this system by adding various concentrations of these substances. The IC₅₀ is defined as the concentration of the particular inhibitor which reduces the formation of angiotensin I by 50%. The compounds of the present invention show inhibitory effects in the in vitro systems at minimal concentrations of about 10⁻⁶ to about 10⁻¹⁹ mol/l.

Illustrative of the invention, the compound of example 501 inhibits the formation of angiotensin I with an IC₅₀ value of 48·10⁻⁹ mol/l.

Renin inhibitors bring about a fall in blood pressure in salt-depleted animals. Human renin differs from renin of other species. Inhibitors of human renin are tested using primates (marmosets, Callithrix jacchus) because human renin and primate renin are substantially homologous in the enzymatically active region. The following in vivo test is employed inter alia: the test compounds are tested on normotensive marmosets of both sexes with a body weight of about 350 g, which are conscious, unrestrained and in their normal cages. Blood pressure and heart rate are measured with a catheter in the descending aorta and are recorded radiometrically. Endogenous release of renin is stimulated by combining a low-salt diet for 1 week with a single intramuscular injection of furosemide (5-(aminosulfonyl)-4-chloro-2-[(2-furanylmethyl)amino]benzoic acid) (5 mg/kg). 16 hours after the furosemide injection, the test substances are administered either directly into the femoral artery by means of a hypodermic needle or as suspension or solution by gavage into the stomach, and their effect on blood pressure and heart rate is evaluated. The compounds of the present invention have a blood pressure-lowering effect in the described in vivo test with i.v. doses of about 0.003 to about 0.3 mg/kg and with oral doses of about 0.3 to about 30 mg/kg.

The blood pressure-reducing effect of the compounds described herein can be tested in vivo using the following protocol:

The investigations take place in 5 to 6-week old, male double transgenic rats (dTGR), which overexpress both human angiotensinogen and human renin and consequently develop hypertension (Bohlender J. et al., J. Am. Soc. Nephrol. 2000; 11: 2056-2061). This double transgenic rat strain was produced by crossbreeding two transgenic strains, one for human angiotensinogen with the endogenous promoter and one for human renin with the endogenous promoter. Neither single transgenic strain was hypertensive. The double transgenic rats, both males and females, develop severe hypertension (mean systolic pressure, approximately 200 mm Hg) and die after a median of 55 days if untreated. The fact that human renin can be studied in the rat is a unique feature of this model. Age-matched Sprague-Dawley rats serve as non-hypertensive control animals. The animals are divided into treatment groups and receive test substance or vehicle (control) for various treatment durations. The applied doses for oral administration may range from 0.5 to 100 mg/kg body weight. Throughout the study, the animals receive standard feed and tap water ad libitum. The systolic and diastolic blood pressure, and the heart rate are measured telemetrically by means of transducers implanted in the abdominal aorta, allowing the animals free and unrestricted movement.

The effect of the compounds described herein on kidney damage (proteinuria) can be tested in vivo using the following protocol:

The investigations take place in 4-week old, male double transgenic rats (dTGR), as described above. The animals are divided into treatment groups and receive test substance or vehicle (control) each day for 7 weeks. The applied doses for oral administration may range from 0.5 to 100 mg/kg body weight. Throughout the study, the animals receive standard feed and tap water ad libitum. The animals are placed periodically in metabolism cages in order to determine the 24-hour urinary excretion of albumin, diuresis, natriuresis, and urine osmolality. At the end of the study, the animals are sacrificed and the kidneys and hearts may also be removed for determining the weight and for immunohistological investigations (fibrosis, macrophage/T cell infiltration, etc.).

The pharmacokinetic properties of the compounds described herein can be tested in vivo using the following protocol:

The investigations take place in pre-catheterized (carotid artery) male rats (300 g±20%) that can move freely throughout the study. The compound is administered intravenously and orally (gavage) in separate sets of animals. The applied doses for oral administration may range from 0.5 to 50 mg/kg body weight; the doses for intravenous administration may range from 0.5 to 20 mg/kg body weight. Blood samples are collected through the catheter before compound administration and over the subsequent 24-hour period using an automated sampling device (AccuSampler, DiLab Europe, Lund, Sweden). Plasma levels of the compound are determined using a validated LC-MS analytical method. The pharmacokinetic analysis is performed on the plasma concentration-time curves after averaging all plasma concentrations across time points for each route of administration. Typical pharmacokinetics parameters to be calculated include: maximum concentration (Cₘₐₓ), time to maximum concentration (tₘₐₓ), area under the curve from 0 hours to the time point of the last quantifiable concentration (AUC₀₋ₜ), area under the curve from time 0 to infinity (AUC_{0-inf}), elimination rate constant (K), terminal half-life (t_{1/2}), absolute oral bioavailability or fraction absorbed (F), clearance (CL), and Volume of distribution during the terminal phase (Vd).

The compounds of the formula (I) and their pharmaceutically acceptable salts can be used as medicines, e.g. in the form of pharmaceutical compositions. The pharmaceutical compositions can be administered enterally, such as orally, e.g. in the form of tablets, lacquered tablets, sugar-coated tablets, hard and soft gelatine capsules, solutions, emulsions or suspensions, nasally, e.g. in the form of nasal sprays, rectally, e.g. in the form of suppositories, or transdermally, e.g. in the form of ointments or patches. However, administration is also possible parenterally, such as intramuscularly or intravenously, e.g. in the form of solutions for injection.

Tablets, lacquered tablets, sugar-coated tablets and hard gelatine capsules can be produced by processing the compounds of the formula (I) and their pharmaceutically acceptable salts with pharmaceutically inert inorganic or organic excipients. Excipients of these types which can be used for example for tablets, sugar-coated tablets and hard gelatine capsules are lactose, maize starch or derivatives thereof, talc, stearic acid or salts thereof etc.

Excipients suitable for soft gelatine capsules are, for example, vegetable oils, waxes, fats, semisolid and liquid polyols etc.

Excipients suitable for producing solutions and syrups are, for example, water, polyols, sucrose, invert sugar, glucose etc.

Excipients suitable for solutions for injection are, for example, water, alcohols, polyols, glycerol, vegetable oils, bile acids, lecithin etc.

Excipients suitable for suppositories are, for example, natural or hardened oils, waxes, fats, semiliquid or liquid polyols etc.

The pharmaceutical compositions may in addition comprise preservatives, solubilizers, viscosity-increasing substances, stabilizers, wetting agents, emulsifiers, sweeteners, colorants, aromatizers, salts to alter the osmotic pressure, buffers, coating agents or antioxidants. They may also comprise other substances of therapeutic value.

The present invention further provides the use of the compounds of the formula (I) and their pharmaceutically acceptable salts in the treatment or prevention of high blood pressure, heart failure, glaucoma, myocardial infarction, renal failure or restenoses.

The compounds of the formula (I) and their pharmaceutically acceptable salts can also be administered in combination with one or more agents having cardiovascular activity, e.g. alpha- and beta-blockers such as phentolamine, phenoxybenzamine, prazosin, terazosin, tolazine, atenolol, metoprolol, nadolol, propranolol, timolol, carteolol etc.; vasodilators such as hydralazine, minoxidil, diazoxide, nitroprusside, flosequinan etc.; calcium antagonists such as aminone, bencyclan, diltiazem, fendiline, flunarizine, nicardipine, nimodipine, perhexiline, verapamil, gallopamil, nifedipine etc.; ACE inhibitors such as cilazapril, captopril, enalapril, lisinopril etc.; potassium activators such as pinacidil; antiserotoninergics such as ketanserine; thromboxane synthetase inhibitors; neutral endopeptidase inhibitors (NEP inhibitors); angiotensin II antagonists; and diuretics such as hydrochlorothiazide, chlorothiazide, acetazolamide, amiloride, bumetanide, benzthiazide, ethacrynic acid, furosemide, indacrinone, metolazone, spironolactone, triamterene, chlorthalidone etc.; sympatholytics such as methyldopa, clonidine, guanabenz, reserpine; and other agents suitable for the treatment of high blood pressure, heart failure or vascular disorders associated with diabetes or renal disorders such as acute or chronic renal failure in humans and animals. Such combinations can be used separately or in products which comprise a plurality of components.

Further substances which can be used in combination with the compounds of formula (I) are the compounds of classes (i) to (ix) on page 1 of WO 02/40007 (and the preferences and examples detailed further therein) and the substances mentioned on pages 20 and 21 of WO 03/027091.

The dosage may vary within wide limits and must of course be adapted to the individual circumstances in each individual case. In general, a daily dose appropriate for oral administration ought to be from about 3 mg to about 3 g, preferably about 10 mg to about 1 g, e.g. approximately 300 mg per adult person (70 kg), divided into preferably 1-3 single doses, which may be for example of equal size, although the stated upper limit may also be exceeded if this proves to be indicated, and children usually receive a reduced dose appropriate for their age and body weight.

### EXAMPLES

The following examples illustrate the present invention. All temperatures are stated in degrees Celsius and pressures in mbar. Unless mentioned otherwise, the reactions take place at RT. The abbreviation “Rf=xx (A)” means for example that the Rf is found in solvent system A to be xx. The ratio of amounts of solvents to one another is always stated in parts by volume. Chemical names for final products and intermediates have been generated on the basis of the chemical structural formulae with the aid of the AutoNom 2000 (Automatic Nomenclature) program.

Thin-layer chromatography element systems:

| | |
|---|---|
| A | Dichloromethane/MeOH/conc. ammonia 25% = 200:20:1 |
| B | Dichloromethane/MeOH/conc. ammonia 25% = 200:20:0.5 |
| C | Dichloromethane/MeOH/conc. ammonia 25% = 200:10:1 |
| D | Dichloromethane/MeOH/conc. ammonia 25% = 90:10:1 |
| E | Dichloromethane/MeOH/conc. ammonia 25% = 60:10:1 |
| F | Dichloromethane/MeOH/conc. ammonia 25% = 200:30:1 |
| G | Dichloromethane/MeOH = 9:1 |
| H | Dichloromethane/MeOH/conc. ammonia 25% = 200:15:1 |

HPLC gradients on Hypersil BDS C-18 (5 um); column: 4×125 mm
(I) 90% water/10% acetonitrile* to 0% water/100% acetonitrile* in 5 minutes+2.5 minutes (1.5 ml/min)
(II) 95% water/5% acetonitrile* to 0% water/100% acetonitrile* in 30 minutes+5 minutes (0.8 ml/min)
*contains 0.1% trifluoroacetic acid

The following abbreviations are used:
AcOH acetic acid
n-BuLi n-butyllithium
t-BuOH tert-butanol
CH₂Cl₂ dichloromethane
CHCl₃ chloroform
CH₃CN acetonitrile
Cy cyclohexane
DCC dicyclohexylcarbodiimide
DIBAL diisobutylaluminium hydride
DME 1,2-dimethoxyethane
DMF N,N-dimethylformamide
EDC.HCl N-ethyl-N′-(3-dimethylaminopropyl)carbodiimide hydrochloride [25952-53-8]
Et₃N triethylamine
Et₂O diethylether
EtOAc ethyl acetate
EtOH ethanol
h hour(s)
HBr hydrobromic acid
HCl hydrochloric acid
H₂O water
K₂CO₃ potassium carbonate
KOH potassium hydroxide
LiCl lithiumchloride
MeI methyl iodide
MeOH methanol
min minute(s)
m.p. melting point (temperature)
N₂ nitrogen
Na₂CO₃ sodium carbonate
NaH sodium hydride
NaHCO₃ sodium bicarbonate
NaOH sodium hydroxide
Na₂SO₄ sodium sulfate
NH₃ ammonia
NH₄Br ammonium bromide
NH₄Cl ammonium chloride
NH₄OH ammonium hydroxide
Pd₂(dba)₃ tris(dibenzylideneacetone)dipalladium [51364-51-3]
Pd(PPh₃)₄ tetrakis-triphenylphosphine palladium(0)
P(tert-Bu)₃ tri-tert-butylphosphine
Ra/Ni Raney-nickel
Rf ratio of distance which a substance travels to distance of the eluent front from the start point in thin layer chromatography
Rt retention time of a substance in HPLC (in minutes)
RT room temperature (23° C.)
TBAF tetrabutylammonium fluoride
TBME tert-butyl methyl ether
TFA trifluoroacetic acid
THF tetrahydrofuran

### Example 500

### (3′S,4′S)-6-(2-Methoxy-ethoxymethyl)-3′-[4-(3-methoxy-propyl)-3,4-dihydro-2H-benzo[1,4]oxazin-6-ylmethoxy]-2-methyl-2′,3′,5′,6′-tetrahydro-1′H-[3,4′]bipyridinyl-4′-ol

To a solution of 0.33 mmol of (3′S,4′S)-4′-hydroxy-6-(2-methoxy-ethoxymethyl)-3′-[4-(3-methoxy-propyl)-3,4-dihydro-2H-benzo[1,4]oxazin-6-ylmethoxy]-2-methyl-3′,4′,5′,6′-tetrahydro-2′H-[3,4′]bipyridinyl-1′-carboxylic acid tert-butyl ester in 2 ml of CH₂Cl₂ is added at 0° C. 6.66 mmol of TFA and the reaction mixture is stirred at RT for 90 min (conversion checked by HPLC or TLC). The reaction mixture is poured into ice-cold saturated aqueous NaHCO₃ (20 ml) and extracted with CH₂Cl₂ (2×100 ml). The combined organic layers are dried over Na₂SO₄ and evaporated. The title compound is obtained from the residue by flash chromatography (SiO₂ 60 F) and is identified based on the Rf value.

The starting material(s) is(are) prepared as follows:
a) (3′S,4′S)-4′-Hydroxy-6-(2-methoxy-ethoxymethyl)-3′-[4-(3-methoxy-propyl)-3,4-dihydro-2H-benzo[1,4]oxazin-6-ylmethoxy]-2-methyl-3′,4′,5′,6′-tetrahydro-2′H-[3,4′]bipyridinyl-1′-carboxylic acid tert-butyl ester

A solution of 1.27 mmol of (3′S,4′S)-4′-hydroxy-6-(2-methoxy-ethoxymethyl)-3′-[4-(3-methoxy-propyl)-3-oxo-3,4-dihydro-2H-benzo[1,4]oxazin-6-ylmethoxy]-2-methyl-3′,4′,5′,6′-tetrahydro-2′H-[3,4′]bipyridinyl-1′-carboxylic acid tert-butyl ester in 22 ml of THF is mixed with 4.0 mmol of borane-THF complex (1M in THF) and stirred at RT for 3 days (conversion checked by HPLC or TLC). After addition of 15 ml of MeOH, the reaction mixture is evaporated. The title compound is obtained from the residue by flash chromatography (SiO₂ 60 F) and is identified based on the Rf value.
b) (3′S,4′S)-4′-Hydroxy-6-(2-methoxy-ethoxymethyl)-3′-[4-(3-methoxy-propyl)-3-oxo-3,4-dihydro-2H-benzo[1,4]oxazin-6-ylmethoxy]-2-methyl-3′,4′,5′,6′-tetrahydro-2′H-[3,4′]bipyridinyl-1′-carboxylic acid tert-butyl ester

7.4 mmol of NaH (60% dispersion in oil) are added to a solution of 6.7 mmol of (3′S,4′S)-3′,4′-dihydroxy-6-(2-methoxy-ethoxymethyl)-2-methyl-3′,4′,5′,6′-tetrahydro-2′H-[3,4′]bipyridinyl-1′-carboxylic acid tert-butyl ester, 7.4 mmol tetrabutylammonium iodide and 7.1 mmol of 6-bromomethyl-4-(3-methoxy-propyl)-4H-benzo[1,4]oxazin-3-one in 25 ml of DMF while the reaction mixture is stirred at 0° C. for 1 h and at RT for 18 h. The mixture is poured into 1M aqueous NaHCO₃ (100 ml) and extracted with CH₂Cl₂ (2×150 ml). The combined organic layers are washed successively with H₂O (2×80 ml) and brine (1×80 ml), dried over Na₂SO₄ and evaporated. Flash chromatography (SiO₂ 60 F) afforded the title compound, which is identified based on the Rf value.
c) (3′S,4′S)-3′,4′-Dihydroxy-6-(2-methoxy-ethoxymethyl)-2-methyl-3′,4′,5′,6′-tetrahydro-2′H-[3,4′]bipyridinyl-1′-carboxylic acid tert-butyl ester

To a stirred solution of (38.3 g) of AD-mix-α [ALDRICH, 39, 275-8, lot 01614BE/277] in 80 ml of t-BuOH and 80 ml of H₂O is added 22.4 mmol of methansulfonamide. The reaction mixture is cooled to 0° C. followed by the addition of 22.4 mmol of 6-(2-methoxy-ethoxymethyl)-2-methyl-3′,6′-dihydro-2′H-[3,4′]bipyridinyl-1′-carboxylic acid tert-butyl ester in 35 ml of t-BuOH and 35 ml of H₂O. The reaction mixture is stirred at 0° C. for 30 min and allowed to stir at RT for 3 days. To the reaction mixture is added 33 g of Na₂SO₃ followed by stirring for 1 h. CH₂Cl₂ (250 ml) is added, the layers are separated and the aqueous layer is extracted again with CH₂Cl₂ (4×150 ml). The combined organic layers are washed with 2N aqueous KOH (200 ml), dried over Na₂SO₄ and concentrated in vacuo. Purification by flash chromatography (SiO₂ 60 F) afforded the title compound, which is identified based on the Rf value.
d) 6-(2-Methoxy-ethoxymethyl)-2-methyl-3′,6′-dihydro-2′H-[3,4′]bipyridinyl-1′-carboxylic acid tert-butyl ester

A three neck flask is charged with 22.2 mmol of 4-trifluoromethane-sulfonyloxy-3,6-dihydro-2H-pyridine-1-carboxylic acid tert-butyl ester [138647-49-1], 30.2 mmol of 3-[6-(2-methoxy-ethoxymethyl)-2-methyl-pyridyl]boronic acid, 66.7 mmol of LiCl, 105 ml of 2N aqueous Na₂CO₃, 220 ml of DME and 1.1 mmol of Pd(PPh₃)₄. The reaction is heated to reflux for 3 h followed by cooling to RT and concentration under reduced pressure. The resulting residue is partitioned between CH₂Cl₂ (500 ml), 2N aqueous Na₂CO₃ (400 ml) and concentrated NH₄OH (25 ml). The layers are separated and the aqueous layer is extracted again with CH₂Cl₂ (3×500 ml). The combined organic layers are dried over Na₂SO₄ and concentrated in vacuo. The resulting black residue is purified by flash chromatography (SiO₂ 60 F) to afford of the title compound, which is identified based on the Rf value.
e) 3-[6-(2-Methoxy-ethoxymethyl)-2-methyl-pyridyl]boronic acid

A solution of 38.8 mmol of n-BuLi (1.6 M in hexanes) is added dropwise to a stirred solution of 32.3 mmol of 3-bromo-6-(2-methoxy-ethoxymethyl)-2-methyl-pyridine in 50 ml of THF at −78° C. The reaction mixture is stirred for 30 min at −78° C. and 64.6 mmol of triisopropyl borate are added rapidly. The mixture is stirred for 30 min at −78° C. and at RT for 1 h. The reaction mixture is partitioned between 2N aqueous HCl (40 ml) and EtOAc (300 ml). The organic layer is washed with brine (2×50 ml), dried over Na₂SO₄ and concentrated in vacuo to afford the title compound, which is identified based on the Rf value.
f) 3-Bromo-6-(2-methoxy-ethoxymethyl)-2-methyl-pyridine

A solution of 1 mmol of 2-methoxy-ethanol [109-86-4] in 2 ml of CH₂Cl₂ is cooled to 0° C. and a solution of 1.2 mmol of 2,2,2-trichloro-acetimidic acid 5-bromo-6-methylpyridin-2-ylmethyl ester in 2 ml of CH₂Cl₂ is added. After 20 min, a solution of 0.075 mmol of trifluoromethane sulfonic acid in 2 ml of CH₂Cl₂ is added dropwise, keeping the inside T at 0° C. The reaction mixture is stirred for 15 min at 0° C., then filtered and diluted with CH₂Cl₂. The residue is admixed with 1M aqueous NaHCO₃ solution (40 ml) and extracted with CH₂Cl₂ (2×60 ml). The organic phases are washed with brine (1×60 ml), dried over Na₂SO₄ and concentrated by evaporation. The title compound is obtained from the residue by means of flash chromatography (SiO₂ 60 F) and identified based on the Rf value.
g) 2,2,2-Trichloro-acetimidic acid 5-bromo-6-methyl-pyridin-2-ylmethyl ester

The solution of 1 mmol of (5-bromo-6-methyl-pyridin-2-yl)-methanol [137778-11-1] in 2.0 ml CH₂Cl₂ is cooled to 0° C. and treated with 1.5 ml of 50% aqueous KOH, followed by 0.05 mmol of tetrabutylammonium hydrogen sulfate. The reaction mixture is stirred at 0° C. for 10 min and then treated dropwise with 1.2 mmol of trichloroacetonitrile, keeping the inside T between 0 and 8° C. The reaction mixture is stirred for 30 min at 0° C. and 30 min at RT. The phases are separated and the aqueous phase is extracted with CH₂Cl₂ (2×20 ml). The combined organic phases are filtered over a plug of silica gel (SiO₂ 60 F) and the solvent is evaporated.

According to the process described in example 500, the following compound is prepared in an analogous manner:
501 (3′S,4′S)-6-((R)-2-Ethoxy-propoxymethyl)-3′-[4-(3-methoxy-propyl)-3,4-dihydro-2H-benzo[1,4]oxazin-6-ylmethoxy]-2-propyl-2′,3′,5′,6′-tetrahydro-1′H-[3,4′]bipyridinyl-4′-ol using 3-[6-((R)-2-ethoxy-propoxymethyl)-2-propyl-pyridyl]boronic acid instead of 3-[6-(2-methoxy-ethoxymethyl)-2-methyl-pyridyl]boronic acid in step d.

The starting material(s) is(are) prepared as follows:
a) 3-[6-((R)-2-Ethoxy-propoxymethyl)-2-propyl-pyridyl]boronic acid

According to example 500e, 3-bromo-6-((R)-2-ethoxy-propoxymethyl)-2-propylpyridine is used to afford the title compound, which is identified based on the Rf value.
b) 3-Bromo-6-((R)-2-ethoxy-propoxymethyl)-2-propyl-pyridine

9.95 mmol of NaH (60% dispersion in oil) are added to a solution of 10.77 mmol of (R)-2-ethoxy-propan-1-ol, 0.83 mmol tetrabutylammonium iodide and 8.29 mmol of 3-bromo-6-chloromethyl-2-propyl-pyridine in 20 ml of DMF while the reaction mixture is stirred at 0° C. for 30 min and at RT for 3 h. The mixture is poured into 1M aqueous NaHCO₃ and extracted with TBME (2×). The combined organic layers are washed successively with H₂O and brine, dried over Na₂SO₄ and evaporated under reduced pressure. The title compound is obtained from the residue by means of flash chromatography (SiO₂ 60 F) and identified based on the Rf value.
c) 3-Bromo-6-chloromethyl-2-propyl-pyridine

4.78 mmol of methanesulfonyl chloride are added to a mixture of 4.35 mmol of (5-bromo-6-propyl-pyridin-2-yl)-methanol, 5.22 mmol of Et₃N and 0.44 mmol of tetrabutylammonium chloride in 40 ml of CH₂Cl₂ at 0° C. and then the reaction mixture is slowly warmed to RT. After a total of 16 h, the reaction is poured into 1M aqueous NaHCO₃ and extracted with CH₂Cl₂ (3×). The combined organic layers are dried over Na₂SO₄ and evaporated under reduced pressure. The title compound is obtained from the residue by means of flash chromatography (SiO₂ 60 F) and identified based on the Rf value.
d) (5-Bromo-6-propyl-pyridin-2-yl)-methanol

A solution of 23.40 mmol of 5-bromo-6-propyl-pyridine-2-carboxylic acid methyl ester in 150 ml of Et₂O is added dropwise to a mixture of 24.57 mmol of lithium aluminium hydride in 150 ml of Et₂O at 0° C. and then the reaction mixture is slowly warmed to RT.

After a total of 16 h, the reaction is poured into saturated aqueous NaHCO₃ and extracted with TBME (3×). The combined organic layers are washed successively with H₂O and brine, dried over Na₂SO₄ and evaporated under reduced pressure. The title compound is obtained from the residue by means of flash chromatography (SiO₂ 60 F) and identified based on the Rf value.
e) 5-Bromo-6-propyl-pyridine-2-carboxylic acid methyl ester

76.20 mmol of trimethylsilyldiazomethane are added dropwise to a solution of 25.40 mmol of 5-bromo-6-propyl-pyridine-2-carboxylic acid in 60 ml of MeOH and 30 ml of heptane at RT. An additional 39.62 mmol of trimethylsilyldiazomethane are added after 1 h, 16 h and 20 h respectively. After a total of 24 h, the reaction mixture is quenched with AcOH and partitioned between saturated aqueous NaHCO₃ and CH₂Cl₂—the aqueous layer is extracted with CH₂Cl₂ (2×). The combined organic layers are dried over Na₂SO₄ and evaporated under reduced pressure. The crude title compound is obtained from the residue and identified based on the Rf value.
f) 5-Bromo-6-propyl-pyridine-2-carboxylic acid

A solution of 25.32 mmol of 5-bromo-6-propyl-pyridine-2-carbonitrile in 85 ml of concentrated HCl is stirred at 90° C. After 66 h, the reaction mixture is cooled to RT and extracted with Et₂O (4×)—the combined organic layers are evaporated under reduced pressure. The crude title compound is obtained from the residue and identified based on the Rf value.
g) 5-Bromo-6-propyl-pyridine-2-carbonitrile

69.89 mmol of Et₃N and then 104.84 mmol of trimethylsilyl cyanide are added to a solution of 34.95 mmol of 3-bromo-2-propyl-pyridine 1-oxide in 45 ml of CH₃CN at RT and then the reaction mixture is heated to 90° C. An additional 52.40 mmol of trimethylsilyl cyanide and 35.03 mmol of Et₃N are added after 16 h and 24 h respectively. After a total of 42 h, the reaction mixture is poured onto a 1:1 mixture of saturated aqueous Na₂CO₃/brine. After separation of the phases, the aqueous phase is extracted with CH₂Cl₂ (3×)—the combined organic layers are dried over Na₂SO₄ and evaporated under reduced pressure. The title compound is obtained from the residue by means of flash chromatography (SiO₂ 60 F) and identified based on the Rf value.
h) 3-Bromo-2-propyl-pyridine 1-oxide

99.28 mmol of 3-chloroperoxybenzoic acid are added portionwise to a solution of 39.71 mmol of 3-bromo-2-propyl-pyridine in 100 ml of CH₂Cl₂ at 0° C. and then the reaction mixture is warmed to RT. After 2 h, the mixture is poured into saturated aqueous NaHCO₃. After separation of the phases, the aqueous phase is extracted with CH₂Cl₂ (3×)—the combined organic layers are washed with saturated aqueous Na₂CO₃, dried over Na₂SO₄ and evaporated under reduced pressure. The crude title compound is obtained from the residue and identified based on the Rf value.
i) 3-Bromo-2-propyl-pyridine

72.53 mmol of manganese(III)acetate dehydrate are added to a mixture of 145.06 mmol of [1-(3-bromo-2-propyl-pyridin-1-yl)-1-phenyl-meth-(E)-ylidene]-methyl-amine in 363 ml of AcOH and then the suspension is heated at 60° C. for 2 h. A solution of 72.53 mmol of periodic acid in 116 ml of 2:1 AcOH/H₂O is added and then the reaction mixture is heated to 80° C. After 1.5 h, the mixture is cooled to RT, diluted with 700 ml of 1N HCl, stirred overnight and then concentrated under reduced pressure to remove AcOH. The residue is partitioned between 2N NaOH and CH₂Cl₂. After separation of the phases, the aqueous phase is extracted with CH₂Cl₂ (2×)—the combined organic layers are dried over Na₂SO₄ and evaporated under reduced pressure. The title compound is obtained from the residue by means of flash chromatography (SiO₂ 60 F) and identified based on the Rf value.
j) [1-(3-Bromo-2-propyl-pyridin-1-yl)-1-phenyl-meth-(E)-ylidene]-methyl-amine

468.02 mmol of 3-bromo-pyridine [626-55-1] are added to a solution of 156.01 mmol of N-methyl-benzamide [613-93-4] in 1400 ml of CH₂Cl₂ and then the mixture is cooled to −40° C. 187.21 mmol of trifluoromethanesulfonic anhydride are added dropwise and then the mixture is warmed to RT. After stirring for 2 h, the reaction mixture is cooled to −78° C. and treated dropwise with 312.01 mmol of propylmagnesium chloride solution (2M in Et₂O). After stirring for an additional 2 h, the mixture is poured into saturated aqueous NaHCO₃ and stirred for 30 min. After separation of the phases, the aqueous phase is extracted with CH₂Cl₂ (2×)—the combined organic layers are dried over Na₂SO₄ and evaporated under reduced pressure. The title compound is obtained from the residue by means of flash chromatography (SiO₂ 60 F) and identified based on the Rf value.
k) (R)-2-Ethoxy-propan-1-ol

17.1 mmol of lithium borohydride are added portionwise to a solution of 11.0 mmol of (R)-2-ethoxy-propionic acid methyl ester in 20 ml of Et₂O under Ar at 0° C. After stirring for 1 h at 0° C. and 18 h at RT, the reaction mixture is slowly poured into an ice-cold saturated aqueous NH₄Cl solution. The phases are separated and then the aqueous phase is extracted with CH₂Cl₂ (5×)—the combined organic phases are dried over Na₂SO₄ and concentrated (35° C., 300 mbar) by evaporation. The crude title compound is obtained from the residue and identified based on the Rf value.
l) (R)-2-Ethoxy-propionic acid methyl ester

28.5 mmol of silver oxide are added to a vigorously stirred solution of 14.25 mmol of methyl-(R)-lactate [17392-83-5] and 28.5 mmol of ethyl iodide in 50 ml of Et₂O under Ar at RT. The reaction flask is wrapped in aluminum foil to exclude light. After 16 h, an additional 14.25 mmol of ethyl iodide and 14.25 mmol of silver oxide are added to the reaction mixture. After 20 h, the reaction is clarified by filtration over Hyflo®, using first Et₂O and then CH₂Cl₂ to wash the filter cake. The combined filtrates are concentrated (35° C., 300 mbar) by evaporation. The title compound is obtained from the residue by means of flash chromatography (SiO₂ 60 F) and identified based on the Rf value.

1. A compound of the general formula (I) 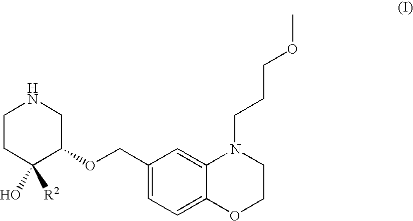
or a pharmaceutically acceptable salt thereof, in which
R² is pyridyl, which is substituted by 1-3 radicals selected independently from the group consisting of
C₁₋₆-alkanoyloxy-C₁₋₆-alkyl,
C₂₋₆-alkenyl,
C₂₋₆-alkenyloxy,
C₂₋₆-alkenyloxy-C₁₋₆-alkyl,
C₁₋₆-alkoxy,
C₁₋₆-alkoxy-C₁₋₆-alkoxy,
C₁₋₆-alkoxy-C₁₋₆-alkoxy-C₁₋₆-alkoxy,
C₁₋₆-alkoxy-C₁₋₆-alkoxy-C₁₋₆-alkoxy-C₁₋₆-alkyl,
C₁₋₆-alkoxy-C₁₋₆-alkoxy-C₁₋₆-alkyl,
C₁₋₆-alkoxy-C₁₋₆-alkyl,
C₁₋₆-alkoxy-C₁₋₆-alkylamino-C₁₋₆-alkyl,
C₁₋₆-alkoxy-C₁₋₆-alkylsulfanyl,
C₁₋₆-alkoxy-C₁₋₆-alkylsulfanyl-C₁₋₆-alkyl,
C₁₋₆-alkoxycarbonyl,
C₁₋₆-alkoxycarbonyloxy-C₁₋₆-alkyl,
C₁₋₆-alkyl,
C₁₋₆-alkylsulfanyl,
C₁₋₆-alkylsulfanyl-C₁₋₆-alkoxy,
C₁₋₆-alkylsulfanyl-C₁₋₆-alkoxy-C₁₋₆-alkyl,
C₁₋₆-alkylsulfanyl-C₁₋₆-alkyl,
C₁₋₆-alkylsulfonyl-C₁₋₆-alkoxy-C₁₋₆-alkyl,
C₁₋₆-alkylsulfonyl-C₁₋₆-alkyl,
C₂₋₈-alkynyl,
optionally N-mono- or N,N-di-C₁₋₆-alkylated amino-C₁₋₆-alkoxy,
optionally N-mono- or N,N-di-C₁₋₆-alkylated amino-carbonyl-C₁₋₆-alkyl,
aryl-pyrrolidinyl-C₀₋₆-alkoxy,
heterocyclyl-pyrrolidinyl-C₀₋₆-alkoxy,
aryloxy,
aryl-C₀₋₆-alkoxy-C₁₋₆-alkoxy,
aryl-C₀₋₆-alkoxy-C₁₋₆-alkoxy-C₁₋₆-alkyl,
carboxy-C₁₋₆-alkyl,
cyano,
cyano-C₁₋₆-alkyl,
C₃₋₈-cycloalkyl-C₀₋₆-alkoxy-C₁₋₆-alkoxy,
C₃₋₈-cycloalkyl-C₀₋₆-alkoxy-C₁₋₆-alkoxy-C₁₋₆-alkyl,
C₃₋₈-cycloalkyl-C₀₋₆-alkoxy-C₁₋₆-alkyl,
C₃₋₈-cycloalkyl-C₀₋₆-alkylamino-C₁₋₆-alkyl,
heterocyclyl-carbonyl-C₁₋₆-alkyl,
heterocyclyl-C₁₋₆-alkyl,
heterocyclyl-sulfanyl-C₁₋₆-alkoxy-C₁₋₆-alkyl and
heterocyclyl-C₀₋₆-alkoxy-C₁₋₆-alkyl;
and may, in addition to the aforementioned substituents, also be substituted by a maximum of 2 halogens, the maximum total number of substituents on the pyridyl radical of R² being 3.

2. The compound according to claim 1, in which R² is pyridyl, substituted by 1-3 radicals selected independently from the group consisting of
C₁₋₆-alkoxy,
C₁₋₆-alkoxy-C₁₋₆-alkoxy,
C₁₋₆-alkoxy-C₁₋₆-alkoxy-C₁₋₆-alkoxy,
C₁₋₆-alkoxy-C₁₋₆-alkoxy-C₁₋₆-alkoxy-C₁₋₆-alkyl,
C₁₋₆-alkoxy-C₁₋₆-alkoxy-C₁₋₆-alkyl,
C₁₋₆-alkoxy-C₁₋₆-alkyl,
C₁₋₆-alkoxy-C₁₋₆-alkylsulfanyl,
C₁₋₆-alkoxy-C₁₋₆-alkylsulfanyl-C₁₋₆-alkyl,
C₁₋₆-alkyl,
C₁₋₆-alkylsulfanyl-C₁₋₆-alkoxy,
C₁₋₆-alkylsulfanyl-C₁₋₆-alkoxy-C₁₋₆-alkyl,
aryl-pyrrolidinyl-C₀₋₆-alkoxy,
C₃₋₈-cycloalkyl-C₀₋₆-alkoxy-C₁₋₆-alkyl,
heterocyclyl-C₀₋₆-alkoxy-C₁₋₆-alkyl and
heterocyclyl-pyrrolidinyl-C₀₋₆-alkoxy, or a pharmaceutically acceptable salt thereof.

3. The compound according to claim 1, in which R² is pyridyl, substituted by 1-2 radicals selected independently from the group consisting of
C₁₋₆-alkoxy,
C₁₋₆-alkoxy-C₁₋₆-alkoxy,
C₁₋₆-alkoxy-C₁₋₆-alkoxy-C₁₋₆-alkoxy,
C₁₋₆-alkoxy-C₁₋₆-alkoxy-C₁₋₆-alkoxy-C₁₋₆-alkyl,
C₁₋₆-alkoxy-C₁₋₆-alkoxy-C₁₋₆-alkyl,
C₁₋₆-alkyl,
C₃₋₈-cycloalkyl-C₀₋₆-alkoxy-C₁₋₆-alkyl,
heterocyclyl-C₀₋₆-alkoxy-C₁₋₆-alkyl and
heterocyclyl-pyrrolidinyl-C₀₋₆-alkoxy, or a pharmaceutically acceptable salt thereof.

4. The compound according to claim 1, in which R² is
pyridyl, substituted by 1 radical selected from the group consisting of
C₁₋₆-alkoxy,
C₁₋₆-alkoxy-C₁₋₆-alkoxy,
C₁₋₆-alkoxy-C₁₋₆-alkoxy-C₁₋₆-alkoxy,
C₁₋₆-alkoxy-C₁₋₆-alkoxy-C₁₋₆-alkoxy-C₁₋₆-alkyl,
C₁₋₆-alkoxy-C₁₋₆-alkoxy-C₁₋₆-alkyl,
C₁₋₆-alkyl,
C₃₋₈-cycloalkyl-C₀₋₆-alkoxy-C₁₋₆-alkyl,
heterocyclyl-C₀₋₆-alkoxy-C₁₋₆-alkyl and
heterocyclyl-pyrrolidinyl-C₀₋₆-alkoxy, or a pharmaceutically acceptable salt thereof.

5. A compound of the general formula (I) or a pharmaceutically acceptable salt thereof, according to any one of claims 1 to 4 for use as a medicine.

6. (canceled)

7. A method for preventing, for delaying the progression of or for treating high blood pressure, heart failure, glaucoma, myocardial infarction, renal failure, restenoses or stroke, where a therapeutically effective amount of a compound of the general formula (I) or of a pharmaceutically acceptable salt thereof, according to any one of claims 1 to 4 is used.

8. A pharmaceutical product comprising a compound of the general formula (I) or of a pharmaceutically acceptable salt thereof, according to any one of claims 1 to 4, and conventional excipients.

9. A pharmaceutical combination in the form of a product or of a kit composed of individual component consisting a) of a compound of the general formula (I) or of a pharmaceutically acceptable salt thereof, according to any one of claims 1 to 4, and b) at least one pharmaceutical form as active ingredient having a cardiovascular effect.
